(19) United States
(12) Patent Application Publication
(10) Pub. No.: US 2006/0024361 A1
(43) Pub. Date: Feb. 2, 2006

(54) Disintegrant assisted controlled release technology

(76) Inventors: Isa Odidi, Toronto (CA); Amina Odidi, Toronto (CA)

Correspondence Address: Dr. Amina Odidi, Suite 106, 405 Britannia Road East, Mississauga, ON, L4Z 3E7, CA

(21) Appl. No.: 10/900,415

(22) Filed: Jul. 28, 2004

(51) Int. Cl. A61K 9/20; A61K 31/7012

(52) U.S. Cl. 424/464; 514/53

(57) ABSTRACT

A disintegrant assisted controlled release device is disclosed. The device is a combination of a swelling disintegrant or super-disintegrant and water insoluble polymer or water soluble polymer, or both, and one or more water soluble or water insoluble active pharmaceutical ingredient(s). The said device is stabilized by a humectant or trehalose.

### FIELD OF THE INVENTION

The present invention provides an improved controlled release device for the delivery of water soluble or water insoluble active pharmaceutical ingredient(s). In particular, the present invention relates to granules, compressed tablets, pellets or capsules consisting of trehalose, a swelling disintegrant or super-disintegrant and water soluble polymer or water insoluble polymer or both, water soluble or water insoluble active pharmaceutical ingredient(s), optionally one or more oil component and optionally silicone dioxide. The swelling disintegrants or super-disintegrants improve and modulate the release of the active pharmaceutical ingredients by the polymers while trehalose is used to stablize the device and superdisintegrants from adverse relative humidity effects which are common with systems containing superdisintegrants. The device may be cured at predetermined temperature and relative humidity for a predetermined period of time in oother to decrease or increase the rate of release of active pharmaceutical ingredients from the device.

The present invention also relates to the controlled release of water soluble or water insoluble active pharmaceutical ingredient(s) in the gastrointestinal tract. The present invention also relates to the use and process of making such granules, tablets, pellets or capsules.

### BACKGROUND OF THE INVENTION

The present invention relates to controlled or sustained release formulations of water soluble or water insoluble active pharmaceutical ingredient(s) that employ a combination of expanding disintegrants or super-disintegrants and water soluble and or water insoluble polymers to control the release of the active pharmaceutical ingredients.

In the prior art, many techniques have been used to provide controlled and sustained-release pharmaceutical dosage forms in order to maintain therapeutic serum levels of medicaments and to minimize the effects of missed doses of drugs caused by a lack of patient compliance and the requirement of decreasing side effects of drugs by controlling their blood concentration.

In the prior art there are extended release tablets which have an osmotically active drug core surrounded by a semipermeable membrane. The semi permeable membrane acts to delimit a reservoir chamber. These tablets function by allowing a fluid such as gastric or intestinal fluid to permeate the coating membrane and dissolve the active ingredient so it can be released through a passageway in the coating membrane by osmotic tension or if the active ingredient is insoluble in the permeating fluid, pushed through the passageway by an expanding agent such as a hydrogel. Some representative examples of these osmotic tablet systems can be found in U.S. Pat. Nos. 3,845,770, 3,916,899, 4,034,758, 4,077,407 and 4,783,337. The problem with these devices is that they are tedious and difficult to fabricate. Their efficiency and precision is also in doubt as they have been known to break up prematurely or retain some of the drug content during transit in the gastrointestinal tract. This may lead to less drug being released and delivered by such devices. It is therefore not uncommon for such devices to contain an overage of drug of at least 10% to account for such inefficiencies in dose delivery. This practice is not economical and presents a danger especially if potent drugs are used, as these devices have been known to rupture in transit thus releasing excess dose.

There have also been reports on sustained-release devices, such as tablets coated with a release-controlling coat, matrix tablets comprising water soluble polymeric compounds, matrix tablets comprising wax, matrix tablets comprising water insoluble polymeric compounds and the like. For example, U.S. Pat. No. 3,629,393 (Nakamoto) utilizes a three-component system to provide slow release tablets in which granules of an active ingredient with a hydrophobic salt of a fatty acid and a polymer are combined with granules of a hydrocolloid and a carrier and granules of a carrier and an active or a buffering agent and then directly compressed into tablets. U.S. Pat. No. 3,728,445 (Bardani) discloses slow release tablets formed by mixing an active ingredient with a solid sugar excipient, granulating the same by moistening with a cellulose acetate phthalate solution, evaporating the solvent, recovering the granules and compressing under high pressure. U.S. Pat. No. 4,704,285 (Alderman) discloses solid slow release tablets containing 5-90% hydroxypropyl cellulose ether, 5-75% of an optional additional hydrophilic colloid such as hydroxypropylmethyl cellulose, an effective amount of an active medicament, and optional binders, lubricants, glidants, fillers, etc.

U.S. Pat. No. 6,605,300 teaches addition of disintegrants to premanufactured drug loaded beads which are to be combined with diluent to make a tablet in order to breakup the tablet and disperse the beads once the tablet is ingested. In this device unlike in the present invention, the disintegrants do not modulate the release of the active pharmaceutical ingredients. They only serve to break up the tablet in order to disperse the beads.

U.S. Pat. No. 6,645,528 teaches Porous drug matrices and methods of manufacture thereof.

These sustained-release devices have difficulty in controlling the release rate of water soluble or water insoluble active pharmaceutical ingredient(s) precisely. It is important that when replacing a multiple times a day dosing with once a day dosing, the loading dose which is represented by the first dose of an immediate release multiple times a day product is captured to a certain extent by the once a day formulation via a loading dose effect which ideally is built into the formulation. Investigational studies over a long period of time were needed to obtain devices with a desired release rate. The desired release rate being a rate of input and extent of release that simulate a loading dose effect and an extended release profile while using a single homogenous unit dose. The difficulty arises because conventional and current controlled release systems require higher amounts of polymers with high molecular weight and viscosity-imparting or gelling properties to achieve true extended release. Unfortunately, such high levels do not result in a loading dose effect. To obtain a loading dose effect in such systems, lower amount of polymer concentration is required or a high amount of water soluble component must be added to moderate the effect of high concentration of polymer. However at these levels high variability is observed within and between lots. It is also difficult to obtain a product with a reproducible release rate and a loading dose effect. Such products also present problems in quality control as precise control and reproducibility of release profiles is difficult.

Therefore it has been strongly desired and sought after to, develop controlled release systems which exhibit a loading dose effect and an extended release profile, while using a single homogenous unit dose, that can be manufactured with excellent reproducibility, and can easily ensure the desired effect of pharmaceutically active components without fail by the administration of one or two times a day.

It is becoming clear that the inclusion of superdisintegrants in systems containing water soluble or water insoluble polymers and water soluble or water insoluble active pharmaceutical ingredient could easily ensure a release rate which is controlled precisely without significant variability according to the purpose, and that products with a reproducible controlled release rate and a loading dose effect could be manufactured. However, this practice is still in its infancy as a search of the prior art will reveal. This is not surprising as laboratory work and testing of devices taught in prior art indicate that systems containing superdisintegrants have stability issues and tend to fail the mandatory stability test set by ICH and the FDA. Superdisintegrants are very moisture sensitive and tend to swell in the presence of humidity resulting in the breakup or at the very least the cracking of the surface of the device. This compromises the said device and adversely alters the original release rate and drug release mechanism built into the device. The shelf life is also affected negatively. This is why to the best of our knowledge there is no commercially available device that utilizes superdisintegrant and water soluble or water insoluble polymers to provide the controlled release of active pharmaceutical ingredients. One way to solve the problem is to use special protective packaging but this is not cost effective.

In view of this situation, the present inventors have undertaken a novel approach and have surprisingly found that the addition of trehalose to systems that combine super-disintegrants, water soluble and or water insoluble polymers and water soluble or water insoluble active pharmaceutical ingredient yield stable systems.

The disclosures in the prior art do not teach the use of humectants or trehalose to stabilize the combination of super-disintegrants and water soluble or water insoluble polymers and optionally an oil component for the controlled or sustained release of water soluble or water insoluble active pharmaceutical ingredient(s). Trehalose dihydrate is stable up to 94% relative humidity. The low hygroscopic nature of trehalose dihydrate results in a free-flowing stable dry product. In food applications where sugars are in the crystalline form, the addition of trehalose can decrease moisture sensitivity and product caking.

### SUMMARY OF THE INVENTION

Accordingly, in view of a need for successfully administering a stable single homogeneous unit controlled release device which controls precisely without significant variability and with a reproducible controlled release rate and a loading dose effect of water soluble or insoluble active pharmaceutical ingredients, the present invention provides a single homogeneous unit controlled release drug delivery system for water soluble or insoluble active pharmaceutical ingredients.

In accordance with a preferred embodiment of the present invention, there is provided a pharmaceutical composition for delivering one or more water soluble or water insoluble active pharmaceutical ingredients consisting of a homogeneous blend of:
(a) one or more water soluble or water insoluble active pharmaceutical ingredients, and
(b) one or more super-disintegrant
(c) one or more water soluble polymers and or water insoluble polymer.
(d) humectant or trehalose
(e) optionally silicone dioxide and one or more oil components

In one embodiment, there is presented a tableting granulated excipient which is free-flowing and directly compressible for use as a controlled release excipient which is a combination of trehalose, one or more super-disintegrant and one or more water soluble and or water insoluble polymer and optionally an inert pharmaceutical filler and silicone dioxide.

Typical conventional controlled release systems using only polymers with super-disintegrants without trehalose do not meet the requirements for a stable single homogeneous unit controlled release device with a good shelf life that can control precisely without significant variability and with a reproducible controlled release rate and a loading dose effect for water soluble or water insoluble active pharmaceutical ingredients. Attempts have been made in the prior art to use water soluble components to modulate the effect of polymers on drug release. These act by creating tortuous channels through which liquid and dissolved drug flows.

These sustained-release devices have difficulty in controlling the desired release rate of water soluble or water insoluble active pharmaceutical ingredient(s) precisely. They fail to capture the loading dose effect which is represented by the first dose of an immediate release multiple times a day product which it is meant to replace.

This has led to scientists such as the inventors to advocate the use of super-disintegrants in combination with water soluble and or water insoluble polymers instead of water soluble components or low amounts of polymers to obtain the desired release rate of input and extent of release that simulate a loading dose effect such that an extended release profile could be achieved using a single homogenous unit dose. This newly emerging field of study indicates that super-disintegrants are able to moderate the negative effect of high concentration of polymer and allow precise control of drug release albeit with the disadvantage that the presence of super-disintegrants introduces stability issues and truncated shelf life.

Typically super-disintegrants present in the device make it reactive to levels of relative humidity that it would otherwise not react to. In a worse case scenerio such devices disintegrate or breakup during storage. This phenomenon is not observed in this invention.

It was surprisingly discovered that the addition of trehalose to the combination of superdisintegrant and polymers impacted on the moisture sensitivity of the preferred embodiment of the present invention. The addition of a humectant or trehalose can decrease moisture sensitivity and enhance product stability.

The drug delivery system of the present invention can be presented as tablets, caplets and pellets for oral, vaginal, anal, ocular, subcutaneous, intramuscular administration or for implantation.

### DETAILED DESCRIPTION OF THE INVENTION

Trehalose is a disaccharide composed of two glucose molecules bound by an alpha, alpha-1, 1 linkage. Since the reducing end of a glucosyl residue is connected with the other, trehalose has no reducing power. Trehalose is widely distributed in nature. It is known to be one of the sources of energy in most living organisms and can be found in many organisms, including bacteria, fungi, insects, plants, and invertebrates. Mushrooms contain up to 10-25% trehalose by dry weight. Furthermore, trehalose protects organisms against various stresses, such as dryness, freezing, and osmopressure. In the case of resurrection plants, which can live in a dry state, when the water dries up, the plants dry up too. However, they can successfully revive when placed in water. The anhydrobitic organisms are able to tolerate the lack of water owing to their ability to synthesize large quantities of trehalose, and the trehalose plays a key role in stabilizing membranes and other macromolecular assemblies under extreme environmental conditions. Trehalose has high thermostability and a wide pH-stability range. Therefore, it is one of the most stable saccharides. Trehalose has a very high glass transition temperature compared to other disaccharides. This allows trehalose to remain stable under a greater range of temperature extremes, providing additional stability to glass systems into which it is incorporated. In addition, trehalose glasses are more resistant to moisture gain than other saccharide glass systems.

Trehalose dihydrate is stable up to 94% relative humidity. The low hygroscopic nature of trehalose dihydrate results in a free-flowing stable dry product. In food applications where sugars are in the crystalline form, the addition of trehalose can decrease moisture sensitivity and product caking.

Water soluble polymers which are used in the present invention may be any polymers which are soluble in water and can retard the release of pharmaceutically active components when made into shapes by press-molding. Preferred water soluble polymers are those which can form hydrocolloid when molded into shape, thereby retarding release of pharmaceutically active components. They include naturally occurring or synthetic, anionic or nonionic, hydrophilic rubbers, starch derivatives, cellulose derivatives, proteins, and the like. Specific examples are acacia, tragacanth, xanthan gum, locust bean gum, guar-gum, karaya gum, pectin, arginic acid, polyethylene oxide, Carbomer, polyethylene glycol, propylene glycol arginate, hydroxypropyl methylcellulose, methylcellulose, hydroxypropyl cellulose, hydroxyethyl cellulose, carboxymethylcellulose sodium, polyvinylpyrrolidone, carboxyvinyl polymer, sodium polyacrylate, alpha starch, sodium carboxymethyl starch, albumin, dextrin, dextran sulfate, agar, gelatin, casein, sodium casein, pullulan, polyvinyl alcohol, deacetylated chitosan, polyethyoxazoline, poloxamers and the like. Of these, preferable are hydroxyethyl cellulose, xanthan gum, hydroxypropyl methylcellulose, methylcellulose, hydroxypropyl cellulose, carbomer, polyethylene glycol, poloxamers, polyethylene oxide, starch derivatives and polyvinylpyrrolidone. These water soluble polymers can be used either singly or in combinations of two or more.

Water insoluble polymers which are used in the present invention may be any polymers which are insoluble in water and can retard the release of pharmaceutically active components. Specific examples of water insoluble polymers are, ethylcellulose, chitin, chitosan, cellulose esters, aminoalkyl methacrylate polymer, anionic polymers of methacrylic acid and methacrylates, copolymers of acrylate and methacrylates with quaternary ammonium groups, ethylacrylate methylmethacrylate copolymers with a neutral ester group, polymethacrylates, surfactants, aliphatic polyesters, zein, polyvinyl acetate, polyvinyl chloride, and the like. Preferred water insoluble polymers are, ethylcellulose, cellulose acetate, polymethacrylates and aminoalkyl methacrylate copolymer.

Oil components which can be used in the current invention include oils and fats, waxes, hydrocarbons, higher fatty acids, higher alcohols, esters, metal salts of higher fatty acids, and the like. Specific examples of oils and fats include plant oils, such as cacao butter, palm oil, Japan wax (wood wax), coconut oil, etc.; animal oils, such as beef tallow, lard, horse fat, mutton tallow, etc.; hydrogenated oils of animal origin, such as hydrogenated fish oil, hydrogenated whale oil, hydrogenated beef tallow, etc.; hydrogenated oils of plant origin, such as hydrogenated rape seed oil, hydrogenated castor oil, hydrogenated coconut oil, hydrogenated soybean oil, etc.; and the like. Of these hydrogenated oils are preferred as an oil component of the present invention. Specific examples of waxes include plant waxes, such as carnauba wax, candelilla wax, bayberry wax, auricurry wax, espalt wax, etc.; animal waxes, such as bees wax, breached bees wax, insect wax, spermaceti, shellac, lanolin, etc.; and the like. Of these preferred are carnauba wax, white beeswax and yellow beeswax. Paraffin, petrolatum, microcrystalline wax, and the like, are given as specific examples of hydrocarbons, with preferable hydrocarbons being paraffin and microcrystalline wax. Given as examples of higher fatty acids are caprilic acid, undecanoic acid, lauric acid, tridecanic acid, myristic acid, pentadecanoic acid, palmitic acid, malgaric acid, stearic acid, nonadecanic acid, arachic acid, heneicosanic acid, behenic acid, tricosanic acid, lignoceric acid, pentacosanic acid, cerotic acid, heptacosanic acid, montanic acid, nonacosanic acid, melissic acid, hentriacontanic acid, dotriacontanic acid, and the like. Of these, preferable are myristic acid, palmitic acid, stearic acid, and behenic acid. Specific examples of higher alcohols are lauryl alcohol, tridecyl alcohol, myristyl alcohol, pentadecyl alcohol, cetyl alcohol, heptadecyl alcohol, stearyl alcohol, nonadecyl alcohol, arachyl alcohol, behenyl alcohol, carnaubic alcohol, corianyl alcohol, ceryl alcohol, and myricyl alcohol. Particularly preferable alcohols are cetyl alcohol, stearyl alcohol, and the like. Specific examples of esters are fatty acid esters, such as myristyl palmitate, stearyl stearate, myristyl myristate, behenyl behenate, ceryl lignocerate, lacceryl cerotate, lacceryl laccerate, etc.; glycerine fatty acid esters, such as lauric monoglyceride, myristic monoglyceride, stearic monoglyceride, behenic monoglyceride, oleic monoglyceride, oleic stearic diglyceride, lauric diglyceride, myristic diglyceride, stearic diglyceride, lauric triglyceride, myristic triglyceride, stearic triglyceride, acetylstearic glyceride, hydoxystearic triglyceride, etc.; and the like. Glycerine fatty acid esters are more preferable. Specific examples of metal salts of higher fatty acid are calcium stearate, magnesium stearate, aluminum stearate, zinc stearate, zinc palmitate, zinc myristate, magnesium myristate, and the like, with preferable higher fatty acid salts being calcium stearate and magnesium stearate.

These oil components and water insoluble polymers can be used either singly or in combinations of two or more.

As used herein, the term “active pharmaceutical ingredients” refers to chemical or biological molecules providing a therapeutic, diagnostic, or prophylactic effect in vivo.

Active pharmaceutical ingredients contemplated for use in the compositions described herein include the following categories and examples of drugs and alternative forms of these drugs such as alternative salt forms, free acid forms, free base forms, and hydrates:

analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, pheneizine, desipramine, nortriptyline, tranylcypromine, fluoxetine, doxepin, imipramine, imipramine pamoate, isocarboxazid, trimipramine, venlafaxine, paroxetine, and protriptyline); antidiabetics (e.g., sulfonylurea derivatives); antifungal agents (e.g., griseofulvin, amphotericin B, nystatin, and candicidin); antihypertensive agents (e.g., propanolol, propafenone, oxyprenolol, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antiteoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, divalproex, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nisoldipine; and nitrates such as nitroglycerin, isosorbide dinitrate, pentaerythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, respiridone, and prochlorperazine); antimanic agents (e.g., lithium carbonate); antiarrhythmics (e.g., bretylium tosylate, esmolol, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillamine, salsalate, piroxicam, azathioprine, indomethacin, meclofenamate, gold sodium thiomalate, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibriolytic agents (e.g., aminocaproic acid); hemorheologic agents (e.g., pentoxifylline): antiplatelet agents (e.g., aspirin); anticonvulsants (e.g., valproic acid, divalproex sodium, phenyloin, phenyloin sodium, clonazepam, primidone, phenobarbitol, amobarbital sodium, methsuximide, metharbital, mephobarbital, mephenyloin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine, loratadine, and); agents useful for calcium regulation (e.g., calcitonin, and parathyroid hormone); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride. cloxacillin sodium, ticarcillin disodium, aziocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin) anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate, steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, tolbutamide, and tolazamide); hypolipidemic agents (e.g., clofibrate, dextrothyroxine sodium, probucol, simvastatin, pravastatin, atorvastatin, lovastatin, and niacin); proteins (e.g., DNase, alginase, superoxide dismutase, and lipase); nucleic acids (e.g., sense or anti-sense nucleic acids encoding any therapeutically useful protein, including any of the proteins described herein); agents useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); oil-soluble vitamins (e.g., vitamins A, D, E, K, and the like); as well as other drugs such as mitotane, halonitrosoureas, anthrocyclines, and ellipticine.

A description of these and other classes of useful drugs and a listing of species within each class can be found in Martindale, The Extra Pharmacopoeia, 30th Ed. (The Pharmaceutical Press, London 1993), the disclosure of which is incorporated herein by reference in its entirety.

Examples of other drugs useful in the compositions and methods described herein include ceftriaxone, ceftazidime, oxaprozin, albuterol, valacyclovir, urofollitropin, famciclovir, flutamide, enalapril, fosinopril, acarbose, lorazepan, follitropin, fluoxetine, lisinopril, tramsdol, levofloxacin, zafirlukast, interferon, growth hormone, interleukin, erythropoietin, granulocyte stimulating factor, nizatidine, perindopril, erbumine, adenosine, alendronate, alprostadil, benazepril, betaxolol, bleomycin sulfate, dexfenfluramine, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, nabumetone, trovafloxacin, dolasetron, zidovudine, finasteride, tobramycin, isradipine, tolcapone, enoxaparin, fluconazole, terbinafine, pamidronate, didanosine, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble.

Other drugs include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, rubitecan, amlodipine besylate/benazepril hydrochloride, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine fumarate, alprostadil, candesartan, cilexetil, fluconazole, ritonavir, busulfan, carbamazepine, flumazenil, risperidone, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, lomefloxacin hydrochloride, tirofiban hydrochloride, telmisartan, diazapam, loratadine, toremifene citrate, thalidomide, dinoprostone, mefloquine hydrochloride, chloroquine, trandolapril, docetaxel, mitoxantrone hydrochloride, tretinoin, etodolac, triamcinolone acetate, estradiol. ursodiol, nelfinavir mesylate, indinavir, beclomethasone dipropionate, oxaprozin, flutamide, famotidine, prednisone, cefuroxime, lorazepam, digoxin, lovastatin, griseofulvin, naproxen, ibuprofen, isotretinoin, tamoxifen citrate, nimodipine, amiodarone, and alprazolam.

Excipients may be selected from diluents, compression agents, extrusion agents, glidants, lubricants, solubilizers, wetting agents, surfactants, penetration enhancers, pigments, colorants, flavoring agents, sweetners, antioxidants, acidulants, stabilizers, antimicrobial preservatives and binders.

These excipients may be chosen from;

(1) diluents such as microcrystalline cellulose, calcium phosphate, mannitol, sorbitol, xylitol, glucitol, ducitol, inositiol, arabinitol; arabitol, galactitol, iditol, allitol, fructose, sorbose, glucose, xylose, trehalose, al lose, dextrose, altrose, gulose, idose, galactose, talose, ribose, arabinose, xylose, lyxose, sucrose, maltose, lactose, lactulose, fucose, rhamnose, melezitose, maltotriose, and raffinose. Preferred sugars include mannitol, lactose, sucrose, sorbitol, trehalose, glucose,

(2) surfactants, wetting agents and solubilisers such as glycerol monostearate, cetostearyl alcohol, cetomacrogol emulsifying wax, sorbitan esters, polyoxyethylene alkyl ethers (e.g., macrogol ethers such as cetomacrogol 1000), polyoxyethlylene castor oil derivatives, polyoxyethylene sorbitan fatty acid esters (e.g., TWEEN™.s), polyoxyethylene stearates, sodium dodecylsulfate, Tyloxapol (a nonionic liquid polymer of the alkyl aryl polyether alcohol type, also known as superinone or triton) is another useful solubilisers. Most of these solubilisers, wetting agents and surfactants are known pharmaceutical excipients and are described in detail in the Handbook of Pharmaceutical Excipients, published jointly by the American Pharmaceutical Association and The Pharmaceutical Society of Great Britain (The Pharmaceutical Press, 1986).

Preferred wetting agents include tyloxapol, poloxamers such as PLURONIC™. F68, F127, and F108, which are block copolymers of ethylene oxide and propylene oxide, and polyxamines such as TETRONIC™. 908 (also known as POLOXAMINE™. 908), which is a tetrafunctional block copolymer derived from sequential addition of propylene oxide and ethylene oxide to ethylenediamine (available from BASF), dextran, lecithin, dialkylesters of sodium sulfosuccinic acid such as AEROSOL™. OT, which is a dioctyl ester of sodium sulfosuccinic acid (available from American Cyanimid), DUPONOL™. P, which is a sodium lauryl sulfate (available from DuPont), TRITON™. X-200, which is an alkyl aryl polyether sulfonate (available from Rohm and Haas), TWEEN™. 20 and TWEEN™. 80, which are polyoxyethylene sorbitan fatty acid esters (available from ICI Specialty Chemicals), Carbowax 3550 and 934, which are polyethylene glycols (available from Union Carbide), Crodesta F-110, which is a mixture of sucrose stearate and sucrose distearate, and Crodesta SL-40 (both available from Croda Inc.), and SA90HCO, which is Cg.sub.18H.sub.37—CH.sub.2 (CON(CH.sub.3)CH.sub.2 (CHOH).sub.4 CF.sub.20H).sub.2.

Wetting agents which have been found to be particularly useful include Tetronic 908, the Tweens, Pluronic F-68 and polyvinylpyrrolidone. Other useful wetting agents include decanoyl-N-methylglucamide; n-decyl-.beta.-D-glucopyranoside; n-decyl-.beta.-D-maltopyranoside; n-dodecyl-.beta.-D-glucopyranoside; n-dodecyl.beta.-D-maltoside; heptanoyl-N-methylglucamide; n-heptyl-.beta.-D-glucopyranoside; n-heptyl-.beta.-D-thioglucoside; n-hexyl-.beta.-D-glucopyranoside; nonanoyl-N-methylglucamide; n-octyl-.beta.-D-glucopyranoside; octanoyl-N-methylglucamide; n-octyl-.beta.-D-glucopyranoside; and octyl-.beta.-D-thioglucopyranoside. Another preferred wetting agent is p-isononylphenoxypoly(glycidol), also known as Olin-10G or Surfactant 10-G (commercially available as 10G from Olin Chemicals). Two or more wetting agents can be used in combination.

In one embodiment, the invention may further include a pegylated excipient. Such pegylated excipients include, but are not limited to, pegylated phospholipids, pegylated proteins, pegylated peptides, pegylated sugars, pegylated polysaccharides, pegylated block-co-polymers with one of the blocks being PEG, and pegylated hydrophobic compounds such as pegylated cholesterol.

Representative examples of pegylated phospholipids include 1,2-diacyl 1-sn-glycero-3-phosphoethanolamine-N-[Poly(ethylene glycol) 2000] (“PEG 2000 PE”) and 1,2-diacyl-sn-glycero-3-phosphoethanolamine-N-[Poly(ethylene glycol) 5000](“PEG 5000 PE”), where the acyl group is selected, for example, from dimyristoyl, dipalmitoyl, distearoyl, diolcoyl, and 1-palmitoyl-2-oleoyl.

One of skill in the art can select appropriate excipients for use in the invention, considering a variety of factors.

There are no specific restrictions as to the methods of manufacture of the controlled release device. It can easily be prepared, for instance, by the dry or wet granulation of a mixture containing trehalose, superdisintegrant, water soluble polymers and or water insoluble polymers, active pharmaceutical ingredients, optionally, an oil component, and optionally, excipients and the like. The granules thus obtained are dried if required and passed through a mill and lubricated.

The controlled release device of the present invention can be prepared according to a conventional method by compressing the granules into a shaped form in rotary tablet press. It can also easily be prepared, by direct compression of a mixture containing trehalose, superdisintegrant, water soluble polymers and or water insoluble polymers, active pharmaceutical ingredients, optionally, an oil component, and optionally, excipients. The controlled release device thus prepared can be used as they are, or further film-coated.

In the controlled release device of the present invention, an ideal release rate for individual pharmaceutically active component can be ensured by controlling its release rate by changing the ratio of trehalose, the super-disintegrant and water soluble and or water soluble polymers and optionally, oil component.

In an embodiment of the present invention the core is coated with a non disintegrating and non semi-permeable coat. Materials useful for forming the non disintegrating non semi-permeable coat are ethylcellulose, polymethylmethacrylates, methacrylic acid copolymers and mixtures thereof.

In yet another embodiment of the present invention the core is coated with a non disintegrating semipermeable coat. Materials useful for forming the non disintegrating semipermeable coat are cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, 4,036,228 and 4,612,008 which are incorporated herein by reference. The most preferred non disintegrating semipermeable coating material is cellulose acetate comprising an acetyl content of 39.3 to 40.3%, commercially available from Eastman Fine Chemicals.

In an alternative embodiment, the non disintegrating semipermeable or non disintegrating non semi-permeable coat can be formed from the above-described polymers and materials that will form passage ways in the coat. The passage way forming agents dissolve on contact with fluid and form passages through which fluid and active pharmaceutical agent can move through the coat. The passage way forming agent can be a water soluble material or an enteric material. Some examples of the preferred materials are sodium chloride, potassium chloride, sucrose, sorbitol, mannitol, polyethylene glycol (PEG), polyvinyl pyrolidone, propylene glycol, hydroxypropyl cellulose, hydroxypropyl methycellulose, hydroxypropyl methycellulose phthalate, cellulose acetate phthalate, polyvinyl alcohols, methacrylic acid copolymers and mixtures thereof. The preferred passage way forming agent is PEG 600, polyvinyl pyrolidone and hydroxypropyl methycellulose.

Active pharmaceutical agents that are water soluble or that are soluble under intestinal conditions may also be used to create passage ways in the coat. The passage way creating agent comprises approximately 0 to about 75% of the total weight of the coating, most preferably about 0.5% to about 25% of the total weight of the coating. The passage way creating agent dissolves or leaches from the coat to form passage ways in the coat for the fluid to enter the core and dissolve the active ingredient.

The coat may also be formed with commonly known excipients such as plasticizer and anti tacking agent. Some commonly known plasticizers include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 20% of the plasticizer can be used based upon the total weight of the coating polymer. The preferred anti tacking agent is talc. Depending on the coating polymer, amounts of from 0 to about 70%, and preferably about 10% to about 50% of talc can be used based upon the total weight of the coating polymer.

As used herein the term passageway includes an aperture, orifice, bore, hole, weaken area or as created by soluble or leachable materials

Generally, the coat around the core will comprise from about 0.5% to about 70% and preferably about 0.5% to about 50% based on the total weight of the core and coating.

In an alternative embodiment, the dosage form of the present invention may also comprise an effective amount of the active pharmaceutical agent that is available for immediate release as a loading dose. This may be coated onto the coat of the dosage form or it may be incorporated into the coat or it may be press coated unto the coated tablet.

In the preparation of the tablets of the invention, various conventional well known solvents may be used to prepare the granules and apply the external coating to the tablets of the invention. In addition, various diluents, excipients, lubricants, dyes, pigments, dispersants etc. which are disclosed in Remington's Pharmaceutical Sciences, 1995 Edition may be used in the invention.

Other features of the invention will become apparent in the course of the following description of the exemplary embodiments which are given for illustration of the invention and are not intended to be limiting thereof.

### EXAMPLE 1

A controlled release tablet containing metoprolol succinate and having the following formula is prepared as follows:

| | % |
|---|---|
| Metoprolol Succinate | 20 |
| Trehalose | 20 |
| Silicone dioxide | 1 |
| Crospovidone | 20 |
| Xanthan gum | 20 |
| Lactose | 18 |
| Magnesium stearate | 1 |

(a) Granulation

The metoprolol succinate, trehalose, silicone dioxide, crospovidone, lactose and Xanthan is added to fluid bed granulator with a top spray assembly. This is granulated by spraying a 1% binding solution of polyvinyl pyrolidone. Once the binding solution is depleted, the granules are dried in the granulator until the loss on drying is less than 5%. The dried granules are passed through a Comil.

(b) Tableting

The magnesium stearate is blended with the granules in a V-blender. After blending, the granules are compressed to tablets on a rotary press.

### EXAMPLE 2

A controlled release tablet containing venlafaxine hydrochloride and having the following formula is prepared as follows:

| | % |
|---|---|
| Venlafaxine hydrochloride | 20 |
| Trehalose | 30 |
| Silicone dioxide | 1 |
| Crospovidone | 20 |
| Xanthan gum | 10 |
| Ethylcellulose | 10 |
| Lactose | 8 |
| Magnesium stearate | 1 |

(a) Granulation

The venlafaxine hydrochloride, trehalose, silicone dioxide, crospovidone, lactose, Ethylcellulose and Xanthan is added to high shear granulator. This is granulated using isopropyl alcohol. The granules are dried in a fluid bed dryer until the loss on drying is less than 5%. The dried granules are passed through a Comil.

(b) Tableting

The magnesium stearate is blended with the granules in a V-blender. After blending, the granules are compressed to caplets on a rotary press.

C) Curing

The tablets are cured by exposing them to a temperature of 40° C. and relative humidity of 70% for 3 mounts

### EXAMPLE 3

A controlled release tablet containing divalproex sodium and having the following formula is prepared as follows:

| | Drug layer (%) |
|---|---|
| Divalproex sodium | 20 |
| Trehalose | 5 |
| Silicone dioxide | 1 |
| Sodium starch glycolate | 30 |
| Hydroxypropyl methyl cellulose | 20 |
| Hydrogenated castor oil | 2 |
| Lactose | 20 |
| Magnesium stearate | 1 |

(a) Preparation by Wet Granulation

Divalproex Na, trehalose, silicone dioxide, sodium starch glycolate, hydroxypropylmethyl cellulose and lactose is granulated in a Hobart low shear mixer using an alcoholic solution of castor oil. The wet granules are dried in a tray dryer oven. The dried granules are lubricated with magnesium stearate in a V-blender.

### EXAMPLE 4

A controlled release tablet containing Nisoldipine and having the following formula is prepared as follows:

| | Drug layer (%) |
|---|---|
| Nisoldipine | 10 |
| Trehalose | 10 |
| Silicone dioxide | 0.5 |
| Croscarmelose Na | 40 |
| Hydroxyethyl cellulose | 25 |
| Lactose | 10 |
| Sodium lauryl sulphate | 9 |
| Magnesium stearate | 0.5 |

Preparation by Direct Compression

Nisoldipine, silicone dioxide, lactose, hydroxyethyl cellulose, trehalose and sodium laury sulphate is dry blended in a high shear granulator. Magnesium stearate is added to the dry blend in a V-blender.

After blending, the dry blended granules from (a) are compressed into tablets.

### EXAMPLE 5

A controlled release tablet containing Paroxetine Hcl and having the following formula is prepared as follows:

| | Drug layer (%) |
|---|---|
| Paroxetine Hcl | 20 |
| Trehalose | 19 |
| Silicone dioxide | 0.5 |
| Crospovidone | 40 |
| Hydroxypropyl methyl cellulose | 10 |
| Xanthan gum | 10 |
| Magnesium stearate | 0.5 |

Preparation by Direct Compression

Crospovidone, silicone dioxide, trehalose, paroxetine hydrochloride, hydroxypropylmethyl cellulose, and xanthan gum is dry blended in a Hobart low shear mixer. Magnesium stearate is added to the dry blend in a V-blender. After blending, the dry blended granules from are compressed into tablets.

### EXAMPLE 6

A controlled release pellets consisting of extruded spheroids containing venlafaxine Hcl and having the following formula is prepared as follows:

| | (%) |
|---|---|
| Carvedilol Hcl | 3 |
| Trehalose | 50 |
| Crospovidone | 20 |
| Microcrystalline cellulose | 13 |
| Polysorbate 80 | 3 |
| Glyceryl monooleate | 3 |
| Xanthan gum | 8 |

(a) Preparation of Extrudate and Spheroids

Venlafaxine Hcl, trehalose, crospovidone, xcipie gum and microcrystalline cellulose is wet granulated in a Hobart low shear mixer. The wet mass is extruded and spheronized.

### EXAMPLE 7

Preparation of controlled release xcipients for use as a direct compressible premanufactured excipients to be used for controlling the release of active pharmaceutical ingredients

| | % |
|---|---|
| Crospovidone | 30 |
| Trehalose | 20 |
| Silicone dioxide | 1 |
| Hydroxypropyl methylcellulose | 10 |
| Xanthan gum | 10 |
| Ethylcellulose | 9 |
| Lactose | 20 |

The materials are dry blended in a v-blender

### EXAMPLE 8

Optional coating systems that may be used to coat products from examples 1 to 6 are as follows:

1. Non disintegrating non semi-permeable Coat type 1

| | % |
|---|---|
| Eudragit NE 30 D | 41.7 |
| Talc | 12.5 |
| Antifoam agent | 0.1 |
| Water | 45.7 |

Talc is added to water to which antifoaming agent has been added while stirring with a high shear mixer. The mixture is added slowly to Eudragit NE 30 D solution and stirred. The coating solution is then sprayed onto the tablets or to a theoretical weight gain of about 5% to 50%.

2. Using non disintegrating non semi-permeable Coat type 2

| | % |
|---|---|
| Ethylcellulose | 80 |
| Hydroxypropylmethylcellulose | 20 |

This is made as a solution in acetone. The coating solution is then sprayed onto the tablets or pellets to a theoretical weight gain of about 2% to about 15%.

3. Non disintegrating non semi-permeable Coat type 3

| | % |
|---|---|
| Eudragit RL 30 D | 46.3 |
| Triethyl citrate | 2.8 |
| Silicone dioxide | 4.2 |
| Antifoam agent | 0.1 |
| Water | 46.6 |

Silicone dioxide is added to water to which antifoaming agent and triethyl citrate has been added while stirring with a high shear mixer. The mixture is added slowly to Eudragit RL 30 D solution and stirred. The coating solution is then sprayed onto the tablets or pellets to a theoretical weight gain of about 3% to about 20%.

4. Non disintegrating non semi-permeable Coat type 4

| | % |
|---|---|
| Eudragit RL 30 D/Eudragit RS 30 D | 46.3 |
| (1:9) | |
| Triethyl citrate | 2.8 |
| Silicone dioxide | 4.2 |
| Antifoam agent | 0.1 |
| Water | 46.6 |

Silicone dioxide is added to water to which antifoaming agent and triethyl citrate has been added while stirring with a high shear mixer. The mixture is added slowly to a mixture of Eudragit RL 30 D and RS 30 D solution and stirred. The coating solution is then sprayed onto the tablets and pellets to a theoretical weight gain of about 3% to about 15%.

5. Using non disintegrating semi-permeable Coat type 1

| | % |
|---|---|
| Cellulose acetate | 80 |
| Triacetin | 5 |
| PEG 600 | 14.5 |
| Red Iron oxide | 0.5 |

The cellulose acetate is dissolved in acetone while stirring with a high shear mixer. The red iron oxide, polyethylene glycol 600 and triacetin are added to the cellulose acetate solution and stirred until a clear solution is obtained. The clear coating solution is then sprayed onto the tablets or pellets to a theoretical weight gain of about 1% to about 15%
6. Using disintegrating coat type 1

The tablets or pellets may be coated with an Opadry® or LustreClear® material or other suitable water-soluble material by first dissolving the opadry material, preferably Opadry Clear, in purified water. The Opadry solution is then sprayed onto the tablets or pellets to a theoretical coating level of about 2% to about 15%.

7. Using disintegrating coat type 2

| | % |
|---|---|
| Eudragit L 30 D | 46.3 |
| Polyethylene glycol 600 | 2.8 |
| Talc | 7.0 |
| Antifoam agent | 0.1 |
| Water | 50.8 |

Talc is added to water to which antifoaming agent and polyethylene glycol 600 has been added while stirring with a high shear mixer. The mixture is added slowly to a mixture of Eudragit L 30 D solution and stirred. The coating solution is then sprayed onto the tablets and pellets to a theoretical weight gain of about 3% to about 15%.

8. Using disintegrating coat type 2

| | % |
|---|---|
| Eudragit L and or Eudragit S | 10.0 |
| Polyethylene glycol 600 | 2.0 |
| Talc | 5.0 |
| Antifoam agent | 0.1 |
| Water | 5.0 |
| Ethanol | 77.9 |

Talc is added to ethanol and water to which antifoaming agent and polyethylene glycol 600 has been added while stirring with a high shear mixer. The mixture is added slowly to a mixture of Eudragit L and or Eudragit S in Ethanol and stirred. The coating solution is then sprayed onto the tablets and pellets to a theoretical weight gain of about 3% to about 20%.

We claim:

1. A solid unit dosage form controlled release device, which comprises at least:
(a) one or more swelling disintegrant or a super-disintegrant, and
(b) one or more water insoluble polymer or water soluble polymer, or both, and
(c) water soluble or water insoluble active pharmaceutical ingredient(s) and
(d) a humectant or trehalose and
(d) optionally silicone dioxide and
(e) optionally one or more oil component

2. The controlled release device according to claim 1, wherein said water-soluble polymer is used in the amount of 1% or more by weight based on the total amount of materials in the device.

3. The controlled release device according to claim 1, wherein said water-insoluble polymer or oil component is used in the amount of 1% or more by weight based on the total amount of materials in the device.

4. The controlled release device according to claim 1, wherein said water-soluble polymer is selected from the group consisting of naturally occurring or synthetic, anionic or nonionic, hydrophilic rubbers, cellulose derivatives, starch derivatives, polysaccharides, hydrogels, gelling agents, gums, alginates, surfactants, polyethylene glycols, polyethylene oxides, polyvinyl alcohols, crosslinked polymers and proteins.

5. The controlled release device according to claim 1, wherein said water-insoluble polymer or oil component is ethylcellulose, chitin, chitosan, cellulose esters, aminoalkyl methacrylate polymer, anionic polymers of methacrylic acid and methacrylates, copolymers of acrylate and methacrylates with quaternary ammonium groups, ethylacrylate methylmethacrylate copolymers with a neutral ester group, polymethacerlates, surfactants, aliphatic polyesters, zein, oils, fats, glycerides, waxes, hydrocarbons, higher fatty acids, higher alcohols, esters, and metal salts of higher fatty acids.

6. The controlled release device according to claim 1, wherein said expanding disintegrant or super-disintegrant is chosen from the group consisting of a cross-linked cellulose, a cross-linked polymer, a cross-linked starch and ion-exchange resin or combination.

7. The controlled release device according to claim 1, wherein said expanding disintegrant or super-disintegrant is chosen from the group consisting of sodium starch glycolate, sodium croscarmellose, homopolymer of cross-linked N-vinyl-2-pyrrolidone, and alginic acid

8. The controlled release device according to claim 1, wherein said swelling disintegrant or super-disintegrant are present in the amount of 1% by weight or more.

9. The controlled release device according to claim 1, wherein the water soluble or water insoluble active pharmaceutical ingredient(s) are present in the amount of 0.001% or more by weight.

10. The controlled release device according to claim 1, wherein said water-soluble polymer is selected from the group consisting of hydroxypropylmethylcellulose, methylcellulose, hydroxypropylcellulose, hydroxyethylcellulose, xanthan gum, carrageenan, carbomer, polyvinylpyrrolidone, locust bean gum, guar-gum, karaya gum, pectin, arginic acid, and propylene glycol arginate.

11. The controlled release device according to claim 1, wherein said water insoluble polymer or oil component is one or more compounds selected from the group consisting of oils and fats, waxes, hydrocarbons, higher fatty acids, higher alcohols, esters, and metal salts of higher fatty acids.

12. The controlled release device according to claim 1, which includes excipients selected from diluents, compression agents, extrusion agents, glidants, lubricants, solubilizers, wetting agents, surfactants, penetration enhancers, pigments, colorants, flavoring agents, sweetners, antioxidants, acidulants, stabilizers, antimicrobial preservatives and binders.

13. A controlled release device containing trehalose which uses swelling disintegrant or super-disintegrant to modulate the release of water soluble or water insoluble active pharmaceutical ingredient(s).

14. A sustained release device containing trehalose which uses swelling disintegrant or super-disintegrant to modulate the release of water soluble or water insoluble active pharmaceutical ingredient(s).

15. A pulsed release device containing trehalose which uses swelling disintegrant or super-disintegrant to modulate the release of water soluble or water insoluble active pharmaceutical ingredient(s).

16. A delayed release device containing trehalose which uses swelling disintegrant or super-disintegrant to modulate the release of water soluble or water insoluble active pharmaceutical ingredient(s).

17. A drug delivery device containing trehalose which uses swelling disintegrant or super-disintegrant to modulate the release of water soluble or water insoluble active pharmaceutical ingredient(s).

18. A drug delivery device containing trehalose which uses swelling disintegrant or super-disintegrant to modulate the release of water soluble or water insoluble active pharmaceutical ingredient(s) in order to produce multiple peaks.

19. A drug delivery device containing trehalose for chronotherapeutic delivery which uses swelling disintegrant or super-disintegrant to modulate the release of water soluble or water insoluble active pharmaceutical ingredient(s).

20. The sustained-release tablet according to claim 4,5 and 17, wherein said wax is carnauba wax, cethyl esters wax, white beeswax or white wax, yellow beeswax or bees wax.

21. The controlled release device according to claim 4, 5, 11 and 17, wherein said oil is a hydrogenated oil and or vegetable oil.

22. The controlled release device according to claim 4, 5 and 17, wherein said hydrocarbon is paraffin and or microcrystalline wax.

23. The controlled release device according to claim 4, 5 and 17 wherein said higher fatty acid is myristic acid, palmitic acid, stearic acid or behenic acid.

24. The controlled release device according to claim 4, 5 and 17 wherein said higher alcohol is cetyl alcohol or stearyl alcohol.

25. The controlled release device according to claim 4, 5 and 17 wherein said esters are glycerine fatty acid esters.

26. The controlled release device according to claim 1, 4, 5, 12 or 17 wherein the device is made by wet or dry granulation of the components and tableted.

27. The controlled release device according to claim 1, 4, 5, 12 or 17 wherein the device is made by direct tableting.

28. The controlled release device according to claim 1, 4, 5, 12 or 17 wherein the device is made by extrusion-spheronization.

29. The controlled release device according to claim 26, 27 and 28 wherein the device is coated by one or more layers of enteric or non-enteric coat or both.

30. The controlled release device according to claim 1 wherein the device is coated by one or more layers of enteric or non-enteric coat or both.

31. The controlled release device according to claim 17 wherein the device is coated by one or more layers of enteric or non-enteric coat or both.

32. The controlled release device according to claim 1 or 17 wherein there is a lag phase prior to the release of water soluble or water insoluble active pharmaceutical ingredient(s).

33. The controlled release device according to claim 1 or 17 for the treatment of hypertension, angina, diabetes, HIV AIDS, pain, depression, psychosis, microbial infections, gastro esophageal reflux disease, impotence, cancer, cardiovascular diseases, gastric/stomach ulcers, blood disorders, nausea, epilepsy, Parkinson's disease, obesity, malaria, gout, asthma, erectile dysfunction, impotence, urinary incontinence, irritable bowel syndrome, ulcerative colitis, smoking, arthritis, rhinitis, Alzheimer's disease, attention deficit disorder, cystic fibrosis, anxiety, insomnia, headache, fungal infection, herpes, hyperglycemia, hyperlipidemia, hypotension, high cholesterol, hypothyroidism, infection, inflammation, mania, menopause, multiple sclerosis, osteoporosis, transplant rejection, schizophrenia, neurological disorders.

34. A method for providing a universal tableting granulated excipient which is free-flowing and directly compressible for controlled release of a water soluble or insoluble therapeutically active medicament comprising mixing an effective amount of said therapeutically active medicament to render a desired therapeutic effect with a premanufactured granulated controlled release excipient comprising from about 1 to about 90 percent by weight of trehalose from about 5 to about 95 percent by weight of a super-disintegrant and about 5 to about 95 percent by weight water soluble polymer and or water insoluble polymer material, and from about 5 to about 70 percent by weight of an inert pharmaceutical filler and silicone dioxide, and thereafter directly compressing the resulting blend to form a tablet.

35. A universal tableting granulated excipient which is free-flowing and directly compressible for controlled release of a water soluble or insoluble therapeutically active medicament comprising blending an effective amount from about 1 to about 90 percent by weight of trehalose from about 5 to about 95 percent by weight of a super-disintegrant and about 5 to about 95 percent by weight water soluble polymer and or water insoluble polymer material, from about 5 to about 75 percent by weight of an inert pharmaceutical filler and from 0 to about 35 percent by weight of silicone dioxide.

36. The controlled release device according to claim 1, 29, 30 or 31 wherein the device is cured at predetermined temperature and relative humidity over predetermined period in other to decrease or increase the rate of release of active pharmaceutical ingredient from the device.
